(19) United States
(12) Patent Application Publication
(10) Pub. No.: US 2022/0392564 A1
(43) Pub. Date: Dec. 8, 2022

(54) System And Methods For Disease Module Detection

(71) Applicant: Northeastern University, Boston, MA (US)

(72) Inventors: Susan Ghiassian, Boston, MA (US); Jörg Menche, Vienna (AT); Amitabh Sharma, Boston, MA (US); Albert-László Barábasi, Brookline, MA (US)

(21) Appl. No.: 17/812,954

(22) Filed: Jul. 15, 2022

Related U.S. Application Data
(63) Continuation of application No. 15/913,826, filed on Mar. 6, 2018.
Application No. 15/913,826 is a continuation of application No. 15/023,582, filed on Mar. 21, 2016, now abandoned.
(60) Provisional application No. 61/881,042, filed on Sep. 23, 2013.

(51) Int. Cl. G16B 5/20; G16B 5/00; G16B 35/00; G16C 20/60

(52) U.S. Cl. CPC G16B 5/20; G16B 5/00; G16B 35/00; G16C 20/60

(57) ABSTRACT

The present disclosure discusses a system and method for disease module detection. More particularly, a protein network and list of seed proteins are provided to the system. The system iteratively selects one or more candidate proteins for inclusion in the list of seed proteins. The system calculates a connectivity factor for each of the connections of the candidate proteins to proteins listed as seed proteins. Responsive to the calculated connectivity factors the system adds one or more of the candidate proteins to list of seed proteins. At the end of the iterative process the list of seed proteins can be indicative of the disease module.

### RELATED APPLICATIONS

This application is a continuation of U.S. application Ser. No. 15/913,826, filed Mar. 6, 2018, which is a continuation of U.S. application Ser. No. 15/023,582, filed Mar. 21, 2016, now abandoned, which is the U.S. National Stage of International Application No. PCT/US2014/056561, filed on Sep. 19, 2014, published in English, which claims the benefit of U.S. Provisional Application No. 61/881,042, filed on Sep. 23, 2013. The entire teachings of the above applications are incorporated herein by reference.

### GOVERNMENT SUPPORT

This invention was made with government support under P50-HG004233 and 1U01HL108630-01 by the National Institutes of Health (NIH), 11645021 and W911NF-12-C-0028 by DARPA, N000141010968 by The Office of Naval Research, and WMDBRBAA07-J-2-0035 and BRBAA08-Per4-C-2-0033 by the Defense Threat Reduction Agency. The government has certain rights in the invention.

### FIELD OF THE DISCLOSURE

This disclosure generally relates to systems and methods for determining networks of genes associated with a disease phenotype. In particular, this disclosure relates to systems and methods for establishing a disease module responsive to a set of seed genes.

### BACKGROUND OF THE DISCLOSURE

Proteins interact within the human interactome to form protein topologies. The patho-biological properties of a disease and its clinical manifestations can be linked to the clusters that the proteins form. To date, the locations of few disease clusters have been located within the interactome, and those disease clusters that have been located are often incomplete.

### BRIEF SUMMARY OF THE DISCLOSURE

According to one aspect of the disclosure, a method for determining a disease cluster includes receiving, by a connectivity module, an indication of a protein network. The protein network can include a plurality of interconnected proteins. The method can also include receiving, by the connectivity module, an indication of a plurality of seed proteins within the protein network that are associated with the disease. Until a criterion is met, the method can iteratively include selecting, by the connectivity module, one or more candidate proteins and calculating a connectivity factor for each of the one or more candidate proteins. The method can further include updating the plurality of seed proteins to include one of the one or more candidate proteins based on the calculated connectivity factor. The method may also include providing, responsive to the satisfactory of the criterion, an indication of a portion of the plurality of interconnected proteins associated with the disease based on the updated list of seed proteins.

In some implementations, the method can also include ranking the connectivity factor for each of the one or more candidate proteins. The method can also include updating the plurality of seed proteins to include a candidate protein from the one or more candidate proteins with the lowest connectivity factor.

In certain implementations, the one or more candidate proteins are connected to at least one of the plurality of seed proteins in the protein network. The one or more candidate proteins can also be connected to the at least one of the plurality of seed proteins through an intermediate protein.

In some implementations, the criterion is a predetermined number of iterations. The method may also include calculating a probability for each connection of the one or more candidate proteins that each connection is connected to one of the plurality of seed proteins.

The method can also include summing, for each of the one or more candidate proteins, the probabilities that each connection of the one or more candidate proteins is connected to one of the plurality of seed proteins. In some implementations, the protein network is a human interactome. The method can further include updating the plurality of seed proteins to include two or more of the one or more candidate proteins.

According to another aspect of the disclosure, a system for determining a disease cluster can include a storage device configured to store an indication of a protein network and an indication of a plurality of seed proteins. The protein network can include a plurality of interconnected proteins. The plurality of seed proteins can be one or more proteins within the protein network that are associated with a disease. The system can also include a connectivity module. The connectivity module can be configured to retrieve the indication of the protein network and the indication of the plurality of seed proteins from the storage device. The connectivity module can further be configured to select one or more candidate proteins. The connectivity module can also calculate a connectivity factor for each of the one or more candidate proteins. The connectivity module may also update the plurality of seed proteins to include one of the one or more candidate proteins based on the calculated connectivity factor for each of the one or more candidate proteins. The connectivity module can also provide an indication of a portion of the plurality of interconnected proteins associated with the disease.

In some implementations, the connectivity module is also configured to rank the connectivity factor for each of the one or more candidate proteins. The connectivity module can also be configured to update the plurality of seed proteins to include a candidate protein from the one or more candidate proteins with the lowest connectivity factor.

In some implementations, one or more candidate proteins can be connected to at least one of the plurality of seed proteins in the protein network. The one or more candidate proteins can be connected to the at least one of the plurality of seed proteins through an intermediate protein.

In some implementations, the criterion is a predetermined number of iterations. The connectivity module can be configured to calculate a probability for each connection of the one or more candidate proteins that each connection is connected to one of the plurality of seed proteins. The connectivity module can also be configured to sum, for each of the one or more candidate proteins, the probabilities that each connection of the one or more candidate proteins is connected to one of the plurality of seed proteins. In some implementations, the connectivity module can be configured to update the plurality of seed proteins to include two or more of the one or more candidate proteins. In some implementations, the protein network is a human interactome.

The details of various embodiments of the disclosure are set forth in the accompanying drawings and the description below.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram illustrating an example network environment including client machines in communication with remote machines.

FIGS. 1B and 1C are block diagrams illustrating example computing devices useful in connection with the methods and systems described herein.

FIG. 2 illustrates an example protein clustering system.

FIG. 3 illustrates an example method for generating a disease cluster using the example protein clustering system illustrated in FIG. 2.

FIG. 4-7 illustrate an example protein network at different steps in the method illustrated in FIG. 3.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein; and

Section B describes embodiments of systems and methods for detecting disease modules.

### A. Computing and Network Environment

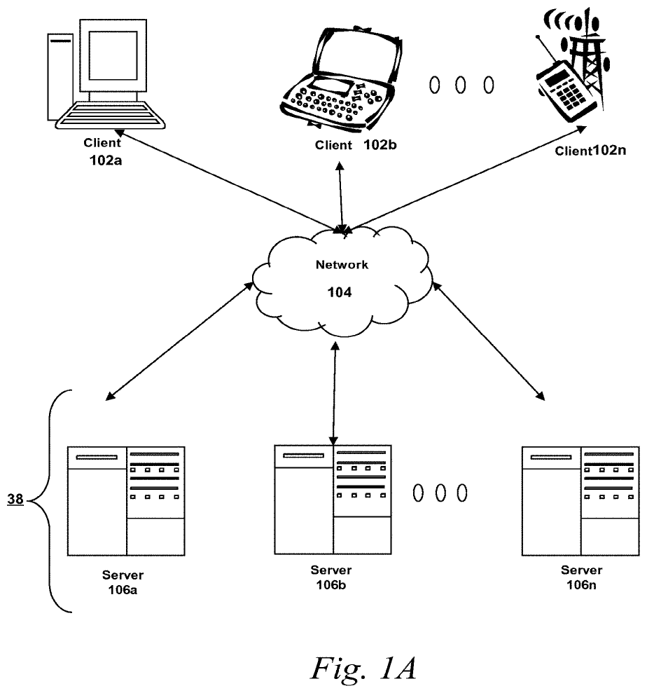

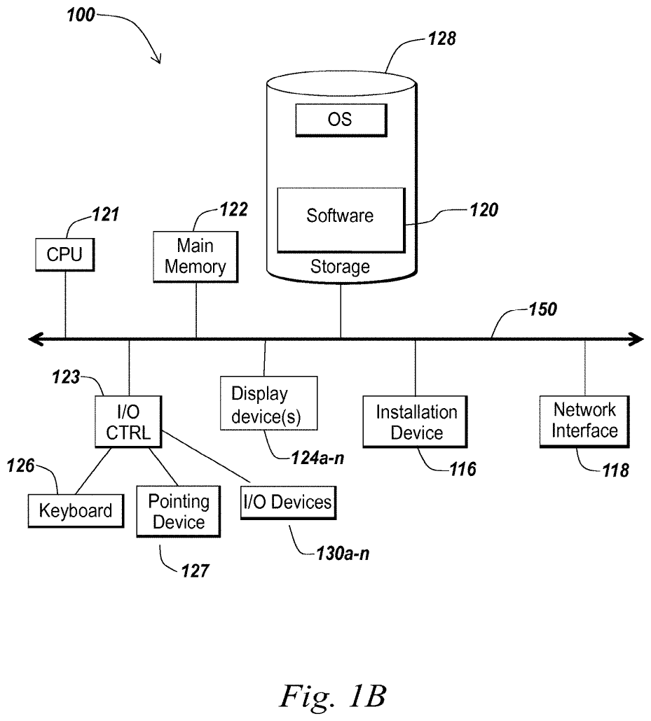

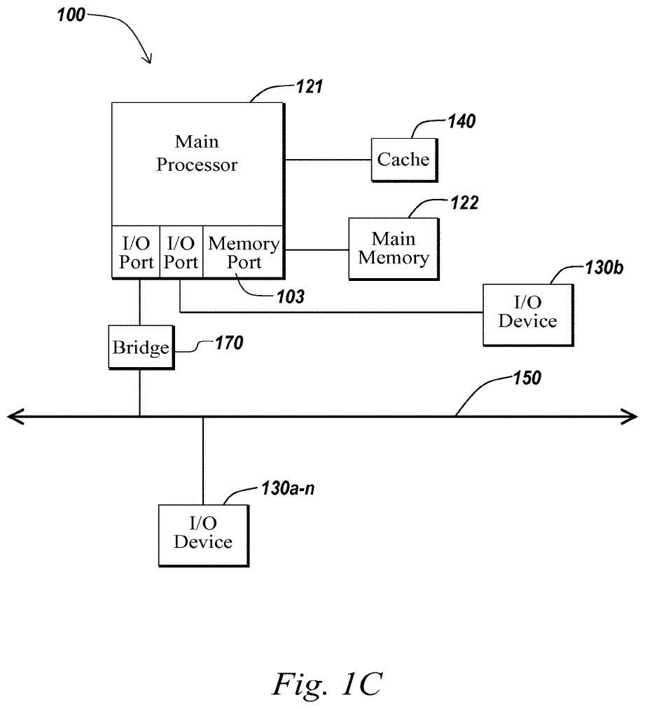

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 101a-101n (also generally referred to as local machine(s) 101, client(s) 101, client node(s) 101, client machine(s) 101, client computer(s) 101, client device(s) 101, endpoint(s) 101, or endpoint node(s) 101) in communication with one or more servers 106a-106n (also generally referred to as server(s) 106, node 106, or remote machine(s) 106) via one or more networks 104. In some embodiments, a client 101 has the capacity to function as both a client node seeking access to resources provided by a server and as a server providing access to hosted resources for other clients 101a-101n.

Although FIG. 1A shows a network 104 between the clients 101 and the servers 106, the clients 101 and the servers 106 may be on the same network 104. The network 104 can be a local-area network (LAN), such as a company Intranet, a metropolitan area network (MAN), or a wide area network (WAN), such as the Internet or the World Wide Web. In some embodiments, there are multiple networks 104 between the clients 101 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be any type and/or form of network and may include any of the following: a point-to-point network, a broadcast network, a wide area network, a local area network, a telecommunications network, a data communication network, a computer network, an ATM (Asynchronous Transfer Mode) network, a SONET (Synchronous Optical Network) network, a SDH (Synchronous Digital Hierarchy) network, a wireless network and a wireline network. In some embodiments, the network 104 may comprise a wireless link, such as an infrared channel or satellite band. The topology of the network 104 may be a bus, star, or ring network topology. The network 104 may be of any such network topology as known to those ordinarily skilled in the art capable of supporting the operations described herein. The network may comprise mobile telephone networks utilizing any protocol(s) or standard(s) used to communicate among mobile devices, including AMPS, TDMA, CDMA, GSM, GPRS, UMTS, WiMAX, 3G or 4G. In some embodiments, different types of data may be transmitted via different protocols. In other embodiments, the same types of data may be transmitted via different protocols.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS, manufactured by Microsoft Corp. of Redmond, Wash.), while one or more of the other servers 106 can operate on according to another type of operating system platform (e.g., Unix or Linux).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments. Hypervisors may include those manufactured by VMWare, Inc., of Palo Alto, Calif.; the Xen hypervisor, an open source product whose development is overseen by Citrix Systems, Inc.; the Virtual Server or virtual PC hypervisors provided by Microsoft or others.

In order to manage a machine farm 38, at least one aspect of the performance of servers 106 in the machine farm 38 should be monitored. Typically, the load placed on each server 106 or the status of sessions running on each server 106 is monitored. In some embodiments, a centralized service may provide management for machine farm 38. The centralized service may gather and store information about a plurality of servers 106, respond to requests for access to resources hosted by servers 106, and enable the establishment of connections between client machines 101 and servers 106.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

In one embodiment, the server 106 provides the functionality of a web server. In another embodiment, the server 106a receives requests from the client 101, forwards the requests to a second server 106b and responds to the request by the client 101 with a response to the request from the server 106b. In still another embodiment, the server 106 acquires an enumeration of applications available to the client 101 and address information associated with a server 106′ hosting an application identified by the enumeration of applications. In yet another embodiment, the server 106 presents the response to the request to the client 101 using a web interface. In one embodiment, the client 101 communicates directly with the server 106 to access the identified application. In another embodiment, the client 101 receives output data, such as display data, generated by an execution of the identified application on the server 106.

The client 101 and server 106 may be deployed as and/or executed on any type and form of computing device, such as a computer, network device or appliance capable of communicating on any type and form of network and performing the operations described herein. FIGS. 1B and 1C depict block diagrams of a computing device 100 useful for practicing an embodiment of the client 101 or a server 106. As shown in FIGS. 1B and 1C, each computing device 100 includes a central processing unit 121, and a main memory unit 122. As shown in FIG. 1B, a computing device 100 may include a storage device 128, an installation device 116, a network interface 118, an I/O controller 123, display devices 124a-101n, a keyboard 126 and a pointing device 127, such as a mouse. The storage device 128 may include, without limitation, an operating system and/or software. As shown in FIG. 1C, each computing device 100 may also include additional optional elements, such as a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, such as: those manufactured by Intel Corporation of Mountain View, Calif.; those manufactured by Motorola Corporation of Schaumburg, Ill.; those manufactured by International Business Machines of White Plains, N.Y.; or those manufactured by Advanced Micro Devices of Sunnyvale, Calif. The computing device 100 may be based on any of these processors, or any other processor capable of operating as described herein.

Main memory unit 122 may be one or more memory chips capable of storing data and allowing any storage location to be directly accessed by the microprocessor 121, such as Static random access memory (SRAM), Burst SRAM or SynchBurst SRAM (BSRAM), Dynamic random access memory (DRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Enhanced DRAM (EDRAM), synchronous DRAM (SDRAM), JEDEC SRAM, PC100 SDRAM, Double Data Rate SDRAM (DDR SDRAM), Enhanced SDRAM (ESDRAM), SyncLink DRAM (SLDRAM), Direct Rambus DRAM (DRDRAM), Ferroelectric RAM (FRAM), NAND Flash, NOR Flash and Solid State Drives (SSD). The main memory 122 may be based on any of the above described memory chips, or any other available memory chips capable of operating as described herein. In the embodiment shown in FIG. 1B, the processor 121 communicates with main memory 122 via a system bus 150 (described in more detail below). FIG. 1C depicts an embodiment of a computing device 100 in which the processor communicates directly with main memory 122 via a memory port 103. For example, in FIG. 1C the main memory 122 may be DRDRAM.

FIG. 1C depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1C, the processor 121 communicates with various I/O devices 130 via a local system bus 150. Various buses may be used to connect the central processing unit 121 to any of the I/O devices 130, including a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 124, the processor 121 may use an Advanced Graphics Port (AGP) to communicate with the display 124. FIG. 1C depicts an embodiment of a computer 100 in which the main processor 121 may communicate directly with I/O device 130b, for example via HYPERTRANSPORT, RAPIDIO, or INFINIBAND communications technology. FIG. 1C also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices include keyboards, mice, trackpads, trackballs, microphones, dials, touch pads, and drawing tablets. Output devices include video displays, speakers, inkjet printers, laser printers, projectors and dye-sublimation printers. The I/O devices may be controlled by an I/O controller 123 as shown in FIG. 1B. The I/O controller may control one or more I/O devices such as a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices such as the USB Flash Drive line of devices manufactured by Twintech Industry, Inc. of Los Alamitos, Calif.

Referring again to FIG. 1B, the computing device 100 may support any suitable installation device 116, such as a disk drive, a CD-ROM drive, a CD-R/RW drive, a DVD-ROM drive, a flash memory drive, tape drives of various formats, USB device, hard-drive or any other device suitable for installing software and programs. The computing device 100 can further include a storage device, such as one or more hard disk drives or redundant arrays of independent disks, for storing an operating system and other related software, and for storing application software programs such as any program or software 120 for implementing (e.g., configured and/or designed for) the systems and methods described herein. Optionally, any of the installation devices 116 could also be used as the storage device. Additionally, the operating system and the software can be run from a bootable medium, for example, a bootable CD.

Furthermore, the computing device 100 may include a network interface 118 to interface to the network 104 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, CDMA, GSM, WiMax and direct asynchronous connections). In one embodiment, the computing device 100 communicates with other computing devices 100′ via any type and/or form of gateway or tunneling protocol such as Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, card bus network adapter, wireless network adapter, USB network adapter, modem or any other device suitable for interfacing the computing device 100 to any type of network capable of communication and performing the operations described herein.

In some embodiments, the computing device 100 may comprise or be connected to multiple display devices 124a-124n, which each may be of the same or different type and/or form. As such, any of the I/O devices 130a-130n and/or the I/O controller 123 may comprise any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may comprise multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices, such as computing devices 100a and 100b connected to the computing device 100, for example, via a network. These embodiments may include any type of software designed and constructed to use another computer's display device as a second display device 124a for the computing device 100. One ordinarily skilled in the art will recognize and appreciate the various ways and embodiments that a computing device 100 may be configured to have multiple display devices 124a-124n.

In further embodiments, an I/O device 130 may be a bridge between the system bus 150 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a FibreChannel bus, a Serial Attached small computer system interface bus, or a HDMI bus.

A computing device 100 of the sort depicted in FIGS. 1B and 1C typically operates under the control of operating systems, which control scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, manufactured by Google Inc; WINDOWS 7 and 8, manufactured by Microsoft Corporation of Redmond, Wash.; MAC OS, manufactured by Apple Computer of Cupertino, Calif.; WebOS, manufactured by Research In Motion (RIM); OS/2, manufactured by International Business Machines of Armonk, N.Y.; and Linux, a freely-available operating system distributed by Caldera Corp. of Salt Lake City, Utah, or any type and/or form of a Unix operating system, among others.

The computer system 100 can be any workstation, telephone, desktop computer, laptop or notebook computer, server, handheld computer, mobile telephone or other portable telecommunications device, media playing device, a gaming system, mobile computing device, or any other type and/or form of computing, telecommunications or media device that is capable of communication. The computer system 100 has sufficient processor power and memory capacity to perform the operations described herein. For example, the computer system 100 may comprise a device of the IPAD or IPOD family of devices manufactured by Apple Computer of Cupertino, Calif., a device of the PLAYSTATION family of devices manufactured by the Sony Corporation of Tokyo, Japan, a device of the NINTENDO/Wii family of devices manufactured by Nintendo Co., Ltd., of Kyoto, Japan, or an XBOX device manufactured by the Microsoft Corporation of Redmond, Wash.

In some embodiments, the computing device 100 may have different processors, operating systems, and input devices consistent with the device. For example, in one embodiment, the computing device 100 is a smart phone, mobile device, tablet or personal digital assistant. In still other embodiments, the computing device 100 is an Android-based mobile device, an iPhone smart phone manufactured by Apple Computer of Cupertino, Calif., or a Blackberry handheld or smart phone, such as the devices manufactured by Research In Motion Limited. Moreover, the computing device 100 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

In some embodiments, the computing device 100 is a digital audio player. In one of these embodiments, the computing device 100 is a tablet such as the Apple IPAD, or a digital audio player such as the Apple IPOD lines of devices, manufactured by Apple Computer of Cupertino, Calif. In another of these embodiments, the digital audio player may function as both a portable media player and as a mass storage device. In other embodiments, the computing device 100 is a digital audio player such as an MP3 players. In yet other embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the communications device 101 includes a combination of devices, such as a mobile phone combined with a digital audio player or portable media player. In one of these embodiments, the communications device 101 is a smartphone, for example, an iPhone manufactured by Apple Computer, or a Blackberry device, manufactured by Research In Motion Limited. In yet another embodiment, the communications device 101 is a laptop or desktop computer equipped with a web browser and a microphone and speaker system, such as a telephony headset. In these embodiments, the communications devices 101 are web-enabled and can receive and initiate phone calls.

In some embodiments, the status of one or more machines 101, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Detecting Disease Modules

The system and methods described herein relate to determining which proteins within a protein network (also referred to as a protein topology or interactome) are associated with a predetermined disease. The system, based on the topology of a protein network and a provided set of proteins known to be associated with the disease (also referred to as seed proteins), can determine which additional proteins within the network are also associated with the disease. The proteins associated with the disease may be referred to as the disease cluster or the disease module. The proteins that are labeled as associated with the disease include the local neighborhood within the protein network that is most likely responsible for the disease phenotype. In some implementations, the creation of the disease module is based on the structure (or connections) within the protein network and requires no other inputs but the seed protein list. Accordingly, the system can be parameter-free. In some implementations, the generated disease modules can be used to identify drug targets, disease pathways and molecular mechanisms, and construct individualized disease modules for personal medicine. The system may be used to determine disease clusters in diseases such, but not limited to, asthma, Ankylosing spondylitis, Celiac Disease, Crohn Disease, Diabetes Mellitus, Graves' Disease, Hashimoto Disease, Lupus, Multiple Sclerosis, Psoriasis, Rheumatoid Arthritis, and Ulcerative Colitis.

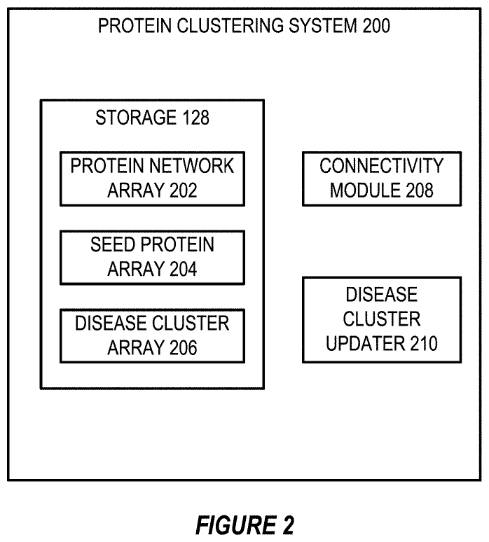

FIG. 2 illustrates an example protein clustering system (PCS) 200. In some implementations, the PCS 200 is a computing device 100, such as the computing device 100 described above in relation to FIG. 1A-1C. In other implementations, the PCS 200 can be implemented by special purpose logic circuitry, such as an FPGA (field programmable gate array) or an ASIC (application specific integrated circuit). The PCS 200 can include a storage device 128 for the storage a protein network array 202, a seed protein array 204, and a disease cluster array 206. The PCS 200 can also include a connectivity module 208 and a disease cluster updater 210.

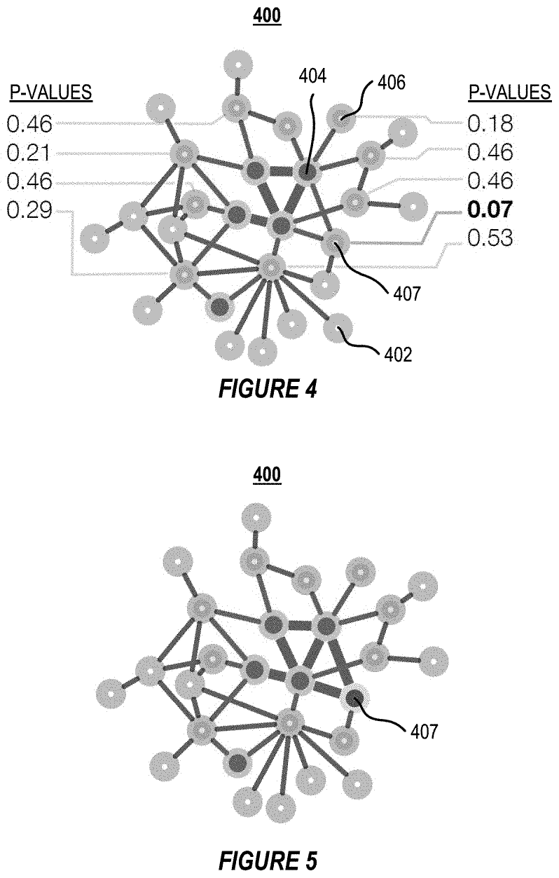

The PCS 200 stores a protein network array 202 within the storage device 128. The protein network array 202 can store data representative of a file, array, or other data source that may be read by the connectivity module 208. The data stored within the protein network array 202 can be data representative of a protein network to be analyzed, which may be referred to as an interactome. In some implementations, the data stored within the protein network array 202 can be referred to as an indication of the protein network or simply the protein network. The protein network can capture the functional interactions between the proteins of the network in a topographical protein map. The protein network may represent the protein (or molecular) interactions that occur within cell. The connections represented within the protein network can represent the physical interactions that may occur between the molecules of the proteins that make up the protein network. A protein network is illustrated and discussed in greater detail in relation to FIG. 4, but in general the protein network can indicate to which proteins each of the proteins within the interactome interact.

In some implementations, the data stored within the protein network array 202 (e.g., the specific protein network to be analyzed by the PCS 200), can be retrieved from a remote server. The protein network may include the Human Interactome, which may be compiled from the regulatory, protein-protein, metabolic, protein complex-based and kinase-substrate interactions that define a human cell's molecular interaction network. In some implementations, the protein network can be curated from scientific literature or downloaded from resources such as, but not limited to, the Human Interactome Project, IntAct, bioGRID, and STRING.

A seed protein array 204 can also be stored within the PCS 200. The seed protein array 204 can store data that indicates which proteins within the protein network that is stored within the protein network array 202 are related to a predetermined disease. For example, the seed protein array 204 may include a list of proteins that are known to be involved causing the predetermined disease. In some implementations, the seed proteins indicated by the seed protein array 204 may be an incomplete list of the proteins within the protein network that are actually associated with the predetermined disease. For example, the list of seed proteins within the seed protein array 204 may include one or more seed proteins truly actually associated with the predetermined disease and may include one or more proteins that are not actually associated with the predetermined disease. In some implementations, the seed protein array 204 is updated during each iteration of the herein described method.

A disease cluster array 206 can also be stored within the PCS 200. The disease cluster array 206 can store a list of proteins that are determined by the PCS 200 to be associated with the predetermined disease. For example, the disease cluster array 206 can be an array the length of the protein network array 202, where every bit in the array corresponds to one of the proteins within the protein network array 202. The bits within the disease cluster array 206 can be flagged when the PCS 200 determines that the specific protein is associated with the predetermined disease. In some implementations, each of the seed proteins stored within the seed protein array 204 are initially also indicated as associated with the predetermined disease cluster by also being stored in the disease cluster array 206. In some implementations, the final output of the method described herein can be distorted in the disease cluster array 206.

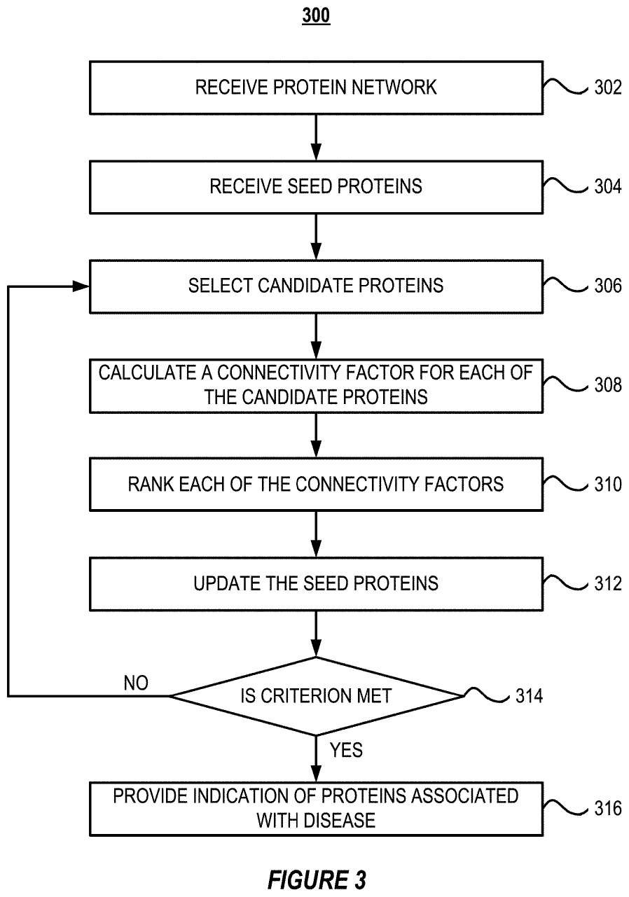

The PCS 200 also includes a connectivity module 208 and a disease cluster updater 210. The connectivity module 208 and the disease cluster updater 210 are discussed in greater detail in relation to FIG. 3. Briefly, the connectivity module 208 can calculate a connectivity factor for each of the proteins within the protein network array 202 that are connected to one of the proteins that are indicated as a seed protein by the seed protein array 204. In some implementations, the connectivity factor indicates the probability that a selected protein in the protein network is connected to one of the seed proteins not by chance. Once the connectivity module 208 has calculated the connectivity factor for each of the proteins in the protein network, the disease cluster updater 210 ranks each of the connectivity factors and determines if any of the proteins should be added to the list of seed proteins (or the disease cluster array) for the next iteration of the calculation made by the connectivity module 208. In some implementations, the connectivity module 208 and the disease cluster updater 210 may include applications, programs, libraries, services, tasks or any type and form of executable instructions that are by one or more processors of the PCS 200.

FIG. 3 illustrates an example method 300 for determining a disease cluster. The method 300 includes retrieving a disease protein network (step 302) and receiving list of seed proteins (step 304). A plurality of candidate proteins are selected (step 306). A connectivity factor for each of the plurality of candidate proteins is calculated (step 308). The calculated connectivity factors are ranked (step 310). Responsive to the ranking of the connectivity factors, the list of seed proteins is updated (step 310). A determination is made whether a criterion is met (step 314). Steps 314 to 312 are repeated until the criterion is met. Responsive to the criterion being met, an indication of the proteins associated with the disease is provided (step 316).

As set forth above, and also referring to FIG. 4, a protein network is provided (step 302). The protein network can be provided as a data file to the PCS 200 or can be manually input into the PCS 200. FIG. 4 illustrates an example protein network 400. The protein network 400 includes a plurality of proteins 402 (also referred to as nodes 402). The proteins 402 of the protein network 400 are interconnected to form a protein topology. Some of the proteins 402 of the protein network 400 can be classified as seed proteins 404 or as candidate proteins. In some implementations, the PCS 200 receives the protein network 400 as a data file, which may be referred to as an indication of the protein network. The data file may indicate the number of proteins 402 within the network 400, which proteins 402 are connected, and the relative strength (or weight) of the connections. The data file may be received as a flat file, a text file, a binary file, an XML file, or a propriety file format. In some implementations, when received by the PCS 200, the PCS 200 may load all or a portion of the protein network 400 into the connectivity module 208. For example, the PCS 200 may load only the proteins within a predetermined distance of the seed proteins rather than loading the entire protein network.

A list of seed proteins is also received (step 302). The received seed protein list can be loaded into the seed protein array 204. Similar, to the received protein network 400, the list of seed proteins can be received as a data file, which may be referred to as an indication of the seed proteins. The data file may be received as a flat file, a text file, a binary file, an XML file, or a propriety file format. Referring again to FIG. 4, the network 400 includes a plurality of seed proteins 404.

One or more candidate proteins can be selected within the protein network (step 306). In some implementations, the candidate proteins can be the proteins that are coupled with one or more of the seed proteins. Referring again to FIG. 4, the candidate proteins 406 are the proteins coupled with one or more of the seed proteins 404. In some implementations, the candidate proteins 406 can be coupled with one or more of the seed proteins by one or two hops. For example, a one-hop candidate protein can be coupled to a seed protein through another protein, which can be referred to as an intermediate protein.

A connectivity factor for each of the candidate proteins can be calculated (step 308). In some implementations, the connectivity factor for each of the candidate proteins indicates the probability or significance that the given candidate protein would be connected to a given seed protein by chance. For some diseases, seed proteins (i.e., proteins associated with a disease) form relatively larger clusters within the protein network than would be expected by chance. Different proteins within a protein network may include a different number of connections to other proteins within the network. For example, in an asthmatic patient IL8 forms 14 connections, of which 4 are known to couple with seed proteins. However, BRCAI makes 239 connections, only 3 of which are to seed proteins. In some implementations, for a protein with a large number of connections, each connection with a seed protein may not be a strong an indication that the protein belongs to the disease cluster. However, for a protein with a relatively small number of total connections each connection to a seed protein can be a strong indication that the protein belongs in the disease cluster. In some implementations, the connectivity factor can be a significance of the number of connections to the seed proteins is calculated to correct for the bias that can occur when the number of connections that each protein makes varies between proteins. In some implementations, the probability that a protein with k connections would be connected to one of the kₛ connections made by the seed proteins by chance is given by the hypergeometric distribution:

$$P ( X = {k}_{s} ) = \frac{( \begin{matrix}s \\ {k}_{s}\end{matrix} ) ( \begin{matrix}N - s \\ k - {k}_{s}\end{matrix} )}{( \begin{matrix}N \\ k\end{matrix} )} . \qquad ( 1 )$$

In equation 1, N denotes the total number of connections in the protein network and s denotes the number of seed proteins in the protein network. The significance of a given number of connections to the seed proteins kₛ can be measured by the p-value:

$$p - value = {\sum }_{n = {k}_{s}}^{k} P ( X = n ) . \qquad ( 2 )$$

In some implementations, the connections between each of the proteins in the protein network can be weighted. For example, the connections made by known seed proteins may be given a higher weight when compared to the seed proteins that are added to the seed protein list (e.g., the seed proteins revealed by the methods described herein). In some implementations, the connections by seed proteins may be given a higher weight when compared to the connections made by non-seed proteins. By considering links to proteins with higher weights to be stronger, the direct neighbors of seed proteins have a higher chance of being identified as part of the disease cluster. Equation 1 can be modified to account for the weights, giving the below equation:

$$P ( X = {k}_{s} ) = \frac{( \begin{matrix}α s \\ α {k}_{s}\end{matrix} ) ( \begin{matrix}N - s \\ k - {k}_{s}\end{matrix} )}{( \begin{matrix}N + ( α - 1 ) {k}_{s} \\ k + ( α - 1 ) {k}_{s}\end{matrix} )} . \qquad ( 3 )$$

In equation 3, a is the weight of the specific protein connection. In some implementations, a for a seed protein can be set between 1 and 20 or between about 5 and 15, where a can be 1 for a non-seed protein.

In some implementations, calculating the p-values can be computationally intensive. In some implementations, the connectivity factor for the proteins can be ranked directly without calculating the p-values for the proteins. In these implementations, proteins with the same k or kₛ values can be ranked based on the respective k or kₛ value. For example, if two candidate proteins have the same k, the candidate protein with the higher kₛ will have fewer terms in equation 2, which results in a lower p-value.

At step 310, each of the connectivity factors are ranked. In some implementations, the connectivity factors are ranked from lowest p-value to highest p-value. A low p-value can indicate that the probability that the protein is connected to the seed protein by chance is low. Referring to FIG. 4, the p-values for each of the candidate proteins 406 are listed.

At step 312, the list of the plurality of seed proteins is updated responsive to the ranking of the candidate proteins from step 310. In some implementations, each of the candidate proteins with a p-value less than a predetermined number may be added to the list of seed proteins. For example, each candidate protein with a p-value less that 0.05 may be added to the list of seed proteins. In some implementations, the candidate protein with the smallest p-value can be added to the list of seed proteins. In the example protein network 400 illustrated in FIG. 4, candidate protein 407 has the lowest p-value, with a p-value of 0.07. FIG. 5 illustrates the protein network 400 at the end of the first iteration. As illustrated, protein candidate 407 has been added to the list of seed proteins. Accordingly, this information may also be reflected within the seed protein array 204 and the disease cluster array 206. For example, a flag indicating that the protein represented by protein 407 is part of the disease cluster may be set and an indication of protein 407 may be added to the list of seed proteins stored in the seed protein array 204. When the protein 407 is added to the seed protein array 204, the s and the kₛ from equation 1 may be appropriately updated. For example, s may be incremented by 1 for the next iteration (s→s+1).

The system may then determine if a criterion is met (step 314). If the criterion is met the method 300 may proceed to step 316. If the criterion is not met the method 300 may return to step 306. In some implementations, the criterion is a predetermined number of iterations. For example, the method 300 may repeat between about 100 times and about 500 times or between about 150 times and about 350 times. In some implementations, the criterion is that no p-value is less than a predetermined threshold. For example, the method 300 may loop until no p-values are less than 0.01. In some implementations, the method may continue until every protein within the protein network is part of the disease cluster (or has been added to the seed protein list). In these implementations, the output of the method described herein may be a ranked list of each of the proteins in the protein network that indicates the likelihood that each of the proteins belongs to the disease cluster.

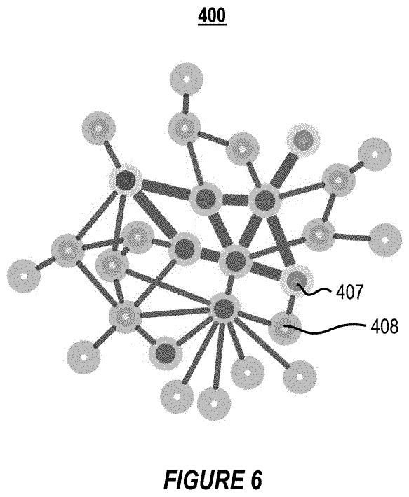

FIG. 6 illustrates the protein network 400 during a second iteration of the method 300. As described above, protein 407 is added to the seed protein list and the method 300 is repeated. During the second iteration of the method 300, protein 408 is included in the list of candidate proteins because protein 408 is connected with protein 407, which is now indicated as a seed protein because it had the lowest p-value in the last iteration. The connectivity factors for each of the new candidate proteins are calculated and then ranked. During the second iteration one or more of the new candidate proteins may be added to the list of seed proteins.

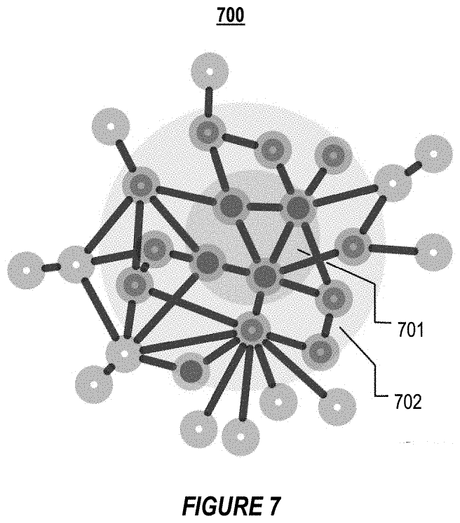

At step 316, responsive to the criterion being met, an indication of the proteins associated with the disease is provided. In some implementations, the indication can be provided to a user in a graphical format, for example as a protein network topology. FIG. 7 illustrates an example output of the method 300. The output protein network 700 indicates the original disease cluster 701, which can correspond to the originally received seed proteins. The output protein network 700 can also indicate the proteins that were added to the seed protein list. The original seed proteins plus the added seed proteins can represent the disease cluster 702. In some implementations, the indication of the proteins associated with the disease is output in as a data file. For example, the data file may be a data file similar to the data files that contained the original protein network data and seed protein data. The data to generate the indication of the disease cluster can come from the seed protein array, the disease cluster array, or a combination thereof.

The present disclosure is not to be limited in terms of the particular embodiments described in this application, which are intended as illustrations of various aspects. Modifications and variations can be made without departing from its spirit and scope of this disclosure. Functionally equivalent methods and apparatuses may exist within the scope of this disclosure. Such modifications and variations are intended to fall within the scope of the appended claims. The subject matter of the present disclosure includes the full scope of equivalents to which it is entitled. This disclosure is not limited to particular methods, reagents, compounds compositions or biological systems, which can vary. The terminology used herein is for the purpose of describing particular embodiments, and is not intended to be limiting.

With respect to the use of substantially any plural or singular terms herein, the plural can include the singular or the singular can include the plural as is appropriate to the context or application.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Claims directed toward the described subject matter may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation can mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction would include but not be limited to systems that have A alone, B alone, C alone, A and B together, A and C together, B and C together, and/or A, B, and C together, etc.). Any disjunctive word or phrase presenting two or more alternative terms, whether in the description, claims, or drawings, can contemplate the possibilities of including one of the terms, either of the terms, or both terms. For example, the phrase “A or B” includes the possibilities of “A” or “B” or “A and B.”

In addition, where features or aspects of the disclosure are described in terms of Markush groups, the disclosure is also described in terms of any individual member or subgroup of members of the Markush group.

Any ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. Language such as “up to,” “at least,” “greater than,” “less than,” and the like include the number recited and refer to ranges which can be subsequently broken down into subranges as discussed above. Finally, a range includes each individual member.

One or more or any part thereof of the techniques described herein can be implemented in computer hardware or software, or a combination of both. The methods can be implemented in computer programs using standard programming techniques following the method and figures described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits preprogrammed for that purpose.

Each such computer program can be stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The analysis, preprocessing, and other methods described herein can also be implemented as a computer-readable storage medium, configured with a computer program, where the storage medium so configured causes a computer to operate in a specific and predefined manner to perform the functions described herein. In some embodiments, the computer readable media is tangible and substantially non-transitory in nature, e.g., such that the recorded information is recorded in a form other than solely as a propagating signal.

In some embodiments, a program product may include a signal bearing medium.

The signal bearing medium may include one or more instructions that, when executed by, for example, a processor, may provide the functionality described above. In some implementations, signal bearing medium may encompass a computer-readable medium, such as, but not limited to, a hard disk drive, a Compact Disc (CD), a Digital Video Disk (DVD), a digital tape, memory, etc. In some implementations, the signal bearing medium may encompass a recordable medium, such as, but not limited to, memory, read/write (R/W) CDs, R/W DVDs, etc. In some implementations, signal bearing medium may encompass a communications medium such as, but not limited to, a digital or an analog communication medium (e.g., a fiber optic cable, a waveguide, a wired communications link, a wireless communication link, etc.). Thus, for example, the program product may be conveyed by an RF signal bearing medium, where the signal bearing medium is conveyed by a wireless communications medium (e.g., a wireless communications medium conforming with the IEEE 802.11 standard).

Any of the signals and signal processing techniques may be digital or analog in nature, or combinations thereof.

While certain embodiments of this disclosure have been particularly shown and described with references to preferred embodiments thereof, various changes in form and details may be made therein without departing from the scope of the disclosure.

We claim:

1. A method of selecting a personalized therapy to a subject suffering from a disease, comprising:
constructing an individualized disease cluster for the subject, comprising:
retrieving a protein network and a list of seed proteins, the protein network comprising a human interactome,
selecting one or more candidate proteins from the protein network,
evaluating a connectivity factor between the one or more candidate proteins and the proteins associated with the list of seed proteins via the protein network, the connectivity factor indicating a probability that a given candidate protein would be connected to a given seed protein by chance, and
classifying one or more candidate proteins having a sufficient connectivity factor as proteins associated with the disease, and wherein the proteins associated with the disease form the individualized disease cluster; and
identifying one or more drug target proteins in the individualized disease cluster for selection of a therapy to which the drug target protein is responsive.

2. The method of claim 1, wherein at least one protein of the list of seed proteins is known to be associated with the disease.

3. The method of claim 1, wherein the connectivity factor between two proteins on the protein network is established by known interactions between two proteins in a cell.

4. The method of claim 1, wherein the one or more candidate proteins are connected directly to one or more proteins from the list of seed proteins on the protein network.

5. The method of claim 1, wherein the one or more candidate proteins are connected via one or more intermediate proteins to one or more proteins from the list of seed proteins on the protein network.

6. The method of claim 1, wherein the one or more candidate proteins are ranked by the connectivity factor.

7. The method of claim 6, wherein at least one ranked candidate protein is added to the list of seed proteins.

8. The method of claim 1, wherein the strength of the connectivity factor is represented by a p-value.

9. The method of claim 8, wherein the candidate protein having the smallest p-value is added to the list of seed proteins.

10. The method of claim 7, further comprising
receiving an updated list of seed proteins;
selecting one or more second candidate proteins from the protein network;
evaluating a connectivity factor between the one or more second candidate proteins and the proteins on the updated list of seed proteins; and
classifying one or more second candidate proteins having a sufficient connectivity factor as proteins associated with the disease, and wherein the proteins associated with the disease form the disease cluster.

11. The method of claim 10, wherein the steps of receiving, selecting, and evaluating are repeated until a criterion is met.

12. The method of claim 11, wherein the criterion is repeating the steps of receiving, selecting, and evaluating a pre-determined number of times.

13. The method of claim 12, wherein the criterion is repeating the steps of receiving, selecting, and evaluating between 100 and 500 times.

14. The method of claim 11, wherein the criterion is that every protein within the protein network is evaluated.

15. The method of claim 1, wherein the disease is selected from asthma, Ankylosing spondylitis, Celiac Disease, Crohn's Disease, Diabetes Mellitus, Graves' Disease, Hashimoto Disease, Lupus, Multiple Sclerosis, Psoriasis, Rheumatoid Arthritis, and Ulcerative Colitis.
